(19) United States
(12) Patent Application Publication
(10) Pub. No.: US 2026/0195915 A1
(43) Pub. Date: Jul. 9, 2026

(54) METHOD AND DEVICE FOR DETECTING MULTI-BRANCH POINT OF BLOOD VESSEL IN MEDICAL IMAGE

(71) Applicant: Medipixel, Inc., Seoul (KR)

(72) Inventors: Junsup SHIN, Seoul (KR); Young-Eon KIM, Seoul (KR)

(21) Appl. No.: 19/441,626

(22) Filed: Jan. 6, 2026

(30) Foreign Application Priority Data
Jan. 7, 2025 (KR) 10-2025-0001890
Mar. 17, 2025 (KR) 10-2025-0034086

(51) Int. Cl. G06T 7/62; A61B 34/10

(52) U.S. Cl. CPC G06T 7/62; A61B 34/10; A61B 2034/107; A61B 2034/108; G06T 2207/10116; G06T 2207/20024; G06T 2207/20092; G06T 2207/30101

(57) ABSTRACT

A technique for detecting a multi-branch point of a blood vessel in a medical image is disclosed. An electronic device may acquire a medical image, extract a target blood vessel, and classify the target blood vessel into first and second blood vessel sections based on branch points of the target blood vessel. The electronic device may determine a target section as a potential multi-branch section by comparing a length between a plurality of branch points in the second blood vessel section with a threshold. The electronic device may generate straight lines intersecting with the target blood vessel at both end points of the potential multi-branch section and at a branch point, and may determine whether the potential multi-branch section is a target multi-branch section based on a position of an intersection between the straight line generated at the branch point and at both end points of the potential multi-branch section.

### CROSS-REFERENCE TO RELATED APPLICATIONS

This application claims the benefit of Korean Patent Application No. 10-2025-0001890, filed on January 7, 2025, and Korean Patent Application No. 10-2025-0034086, filed on March 17, 2025, in the Korean Intellectual Property Office, the entire disclosures of which are incorporated herein by reference for all purposes.

### BACKGROUND

### 1. Field of the Invention

One or more embodiments relate to a technique for detecting a multi-branch point of a target blood vessel in a medical image.

### 2. Description of the Related Art

Angiography is a diagnostic procedure that visualizes blood vessels and their conditions using X-rays and is a useful tool for examining vascular diseases. In angiography, which is useful for diagnosing vascular diseases, accurately identifying the size, shape, and structure of blood vessels is important for determining the severity of diseases and selecting appropriate treatment methods. In particular, when a blood vessel branches into multiple paths, a device for treating the blood vessel can be appropriately selected by accurately identifying the structure of the branch point. To this end, it is necessary to accurately identify a branch point of a blood vessel in a blood vessel image.

The above description has been possessed or acquired by the inventor(s) in the course of conceiving the present disclosure and is not necessarily an art publicly known before the present application is filed.

### SUMMARY

According to an aspect, there is provided an electronic device for medical image processing, the electronic device including an image acquisition unit configured to acquire a medical image, memory configured to store the medical image and a plurality of instructions, and a processor configured to execute the plurality of instructions included in the memory, wherein the processor may extract a target blood vessel from the medical image, classify the target blood vessel into a plurality of first blood vessel sections and a plurality of second blood vessel sections including at least one branch point, determine a target section including a first branch point and a second branch point different from the first branch point among the plurality of second blood vessel sections as a potential multi-branch section based on a first target length between the first branch point and the second branch point, determine the potential multi-branch section as a target multi-branch section based on an intersection point between a first straight line intersecting with the target blood vessel at a predetermined blood vessel point of the target section and a second straight line intersecting with the target blood vessel at the second branch point, and provide vascular information corresponding to each of a plurality of paths connected to the target multi-branch section.

The processor may determine a point adjacent to a heart to which the target blood vessel is connected among both end points of the target section as the predetermined blood vessel point, and determine the target section as the potential multi-branch section based on a result of comparing the first target length to a threshold length calculated based on a vascular diameter at the predetermined blood vessel point.

The processor may obtain the threshold length based on a result of an operation between the vascular diameter at the predetermined blood vessel point and a predetermined threshold value, classify the target section as the second blood vessel section when the first target length is equal to or greater than the threshold length, and determine the target section as the potential multi-branch section when the first target length is less than the threshold length.

The processor may classify the plurality of first blood vessel sections and the second blood vessel sections as a tree structure, obtain nodes of a higher level than a node corresponding to the target section in the tree structure, generate the first straight line at each of predetermined blood vessel points corresponding to points in contact with the target section among both end points of each of first blood vessel sections corresponding to the obtained nodes, and determine the potential multi-branch section as the target multi-branch section based on the intersection point between the first straight line and the second straight line.

The processor may determine whether the intersection point between the first straight line and the second straight line is located within the target blood vessel, determine the potential multi-branch section as the second blood vessel section when the intersection point is located outside the target blood vessel, and determine the potential multi-branch section as the target multi-branch section when the intersection point is located within the target blood vessel.

The processor may, when the target section includes the first branch point, the second branch point located at a distal portion of the target blood vessel relative to the first branch point, and a third branch point located at a distal portion of the target blood vessel relative to the second branch point, determine the target section as the potential multi-branch section based on a second target length between the second branch point and the third branch point, and determine the potential multi-branch section as the target multi-branch section based on an intersection point between the first straight line and a third straight line intersecting with the target blood vessel at the third branch point.

The processor may classify the plurality of first blood vessel sections and the second blood vessel sections as a tree structure.

The processor may provide stent information including at least one of information regarding a number of stents to be inserted into the plurality of paths and information regarding sizes of the stents, based on the vascular information corresponding to each of the plurality of paths connected to the target multi-branch section.

The electronic device according to an embodiment may further include an actuator, wherein the processor may control stent movement in the target multi-branch section by operating the actuator based on the vascular information corresponding to each of the plurality of paths connected to the target multi-branch section.

The processor may receive a predetermined first point and a predetermined second point on the target blood vessel, identify a branch point at which the target blood vessel branches to generate a plurality of blood vessel paths by scanning the target blood vessel from the first point toward the second point, and classify the target blood vessel into the plurality of first blood vessel sections and the second blood vessel sections based on parallel scanning of the plurality of blood vessel paths.

The processor may extract a vascular centerline corresponding to the target blood vessel, detect points at which the vascular centerline branches as branch points while scanning the vascular centerline from a predetermined first point to a predetermined second point, and classify a section including at least one branch point among the branch points as the second blood vessel section.

According to another aspect, there is provided a method of processing a medical image, performed by an electronic device, the method including extracting a target blood vessel from a medical image, classifying the target blood vessel into a plurality of first blood vessel sections and a plurality of second blood vessel sections including at least one branch point, determining a target section including a first branch point and a second branch point different from the first branch point among the plurality of second blood vessel sections as a potential multi-branch section based on a first target length between the first branch point and the second branch point, determining the potential multi-branch section as a target multi-branch section based on an intersection point between a first straight line intersecting with the target blood vessel at a predetermined blood vessel point of the target section and a second straight line intersecting with the target blood vessel at the second branch point, and providing vascular information corresponding to each of a plurality of paths connected to the target multi-branch section.

The determining of the target section as the potential multi-branch section may include determining a point adjacent to a heart to which the target blood vessel is connected among both end points of the target section as the predetermined blood vessel point, and determining the target section as the potential multi-branch section based on a result of comparing the first target length to a threshold length calculated based on a vascular diameter at the predetermined blood vessel point.

The determining of the target section as the potential multi-branch section may include obtaining the threshold length based on a result of an operation between the vascular diameter at the predetermined blood vessel point and a predetermined threshold value, classifying the target section as the second blood vessel section when the first target length is equal to or greater than the threshold length, and determining the target section as the potential multi-branch section when the first target length is less than the threshold length.

The determining of the potential multi-branch section as the target multi-branch section may include determining whether the intersection point between the first straight line and the second straight line is located within the target blood vessel, determining the potential multi-branch section as the second blood vessel section when the intersection point is located outside the target blood vessel, and determining the potential multi-branch section as the target multi-branch section when the intersection point is located within the target blood vessel.

The determining of the potential multi-branch section as the target multi-branch section may include, when the target section includes the first branch point, the second branch point located at a distal portion of the target blood vessel relative to the first branch point, and a third branch point located at a distal portion of the target blood vessel relative to the second branch point, determining the target section as the potential multi-branch section based on a second target length between the second branch point and the third branch point, and determining the potential multi-branch section as the target multi-branch section based on an intersection point between the first straight line and a third straight line intersecting with the target blood vessel at the third branch point.

The classifying of the target blood vessel into the plurality of first blood vessel sections and the plurality of second blood vessel sections may include classifying the plurality of first blood vessel sections and the second blood vessel sections as a tree structure.

The providing of the vascular information may include providing stent information including at least one of information regarding a number of stents to be inserted into the plurality of paths and information regarding sizes of the stents, based on the vascular information corresponding to each of the plurality of paths connected to the target multi-branch section.

The providing of the vascular information may include providing information regarding movement of a stent to be inserted into the target multi-branch section based on the vascular information corresponding to each of the plurality of paths connected to the target multi-branch section.

Additional aspects of embodiments will be set forth in part in the description which follows and, in part, will be apparent from the description, or may be learned by practice of the disclosure

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a block diagram of an electronic device according to an embodiment.

FIG. 2 schematically illustrates a branch type included in a target blood vessel according to an embodiment.

FIG. 3 schematically illustrates a branch type included in a target blood vessel according to an embodiment.

FIG. 4 schematically illustrates a branch type included in a target blood vessel according to an embodiment.

FIG. 5 schematically illustrates a branch type included in a target blood vessel according to an embodiment.

FIG. 6 illustrates a method by which an electronic device classifies a target blood vessel into a plurality of blood vessel sections according to an embodiment.

FIG. 7 illustrates a method by which an electronic device classifies a target blood vessel into a plurality of blood vessel sections according to an embodiment.

FIG. 8 is a diagram for describing a method by which an electronic device determines a potential multi-branch section according to an embodiment.

FIG. 9 is a diagram for describing a method by which an electronic device determines a potential multi-branch section according to an embodiment.

FIG. 10 is a diagram for describing a method by which an electronic device determines a potential multi-branch section according to an embodiment.

FIG. 11 is a diagram for schematically describing a method by which an electronic device determines a target multi-branch section according to an embodiment.

FIG. 12 illustrates a specific method by which an electronic device determines a target multi-branch section in a target blood vessel according to an embodiment.

FIG. 13 is a flowchart schematically illustrating a method performed by an electronic device according to an embodiment.

### DETAILED DESCRIPTION

The following detailed structural or functional description is provided as an example only and various alterations and modifications may be made to the examples. Accordingly, the embodiments are not construed as limited to the disclosure and should be understood to include all changes, equivalents, and replacements within the idea and the technical scope of the disclosure.

Although terms, such as first, second, and the like, are used to describe various components, the components are not limited to the terms. These terms should be used only to distinguish one component from another component. For example, a first component may be referred to as a second component, and similarly the second component may also be referred to as the first component.

It should be noted that if one component is described as being "connected", "coupled", or "joined" to another component, a third component may be "connected", "coupled", and "joined" between the first and second components, although the first component may be directly connected, coupled, or joined to the second component.

As used herein, the singular forms "a," "an," and "the" are intended to include the plural forms as well, unless the context clearly indicates otherwise. It will be further understood that the terms "comprises/comprising" and/or "includes/including" when used herein, specify

the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, the embodiments will be described in detail with reference to the accompanying drawings. When describing the embodiments with reference to the accompanying drawings, like reference numerals refer to like components and a repeated description related thereto will be omitted.

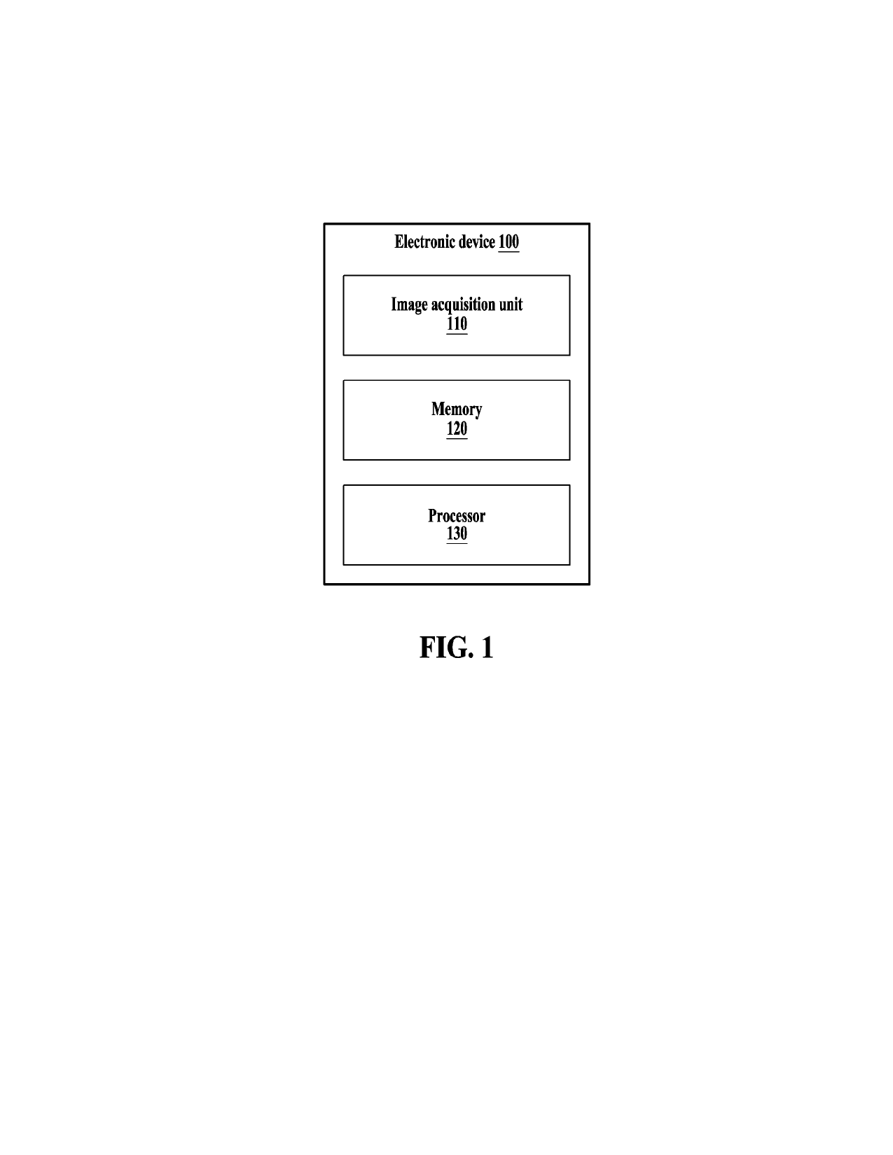

FIG. 1 is a block diagram of an electronic device according to an embodiment.

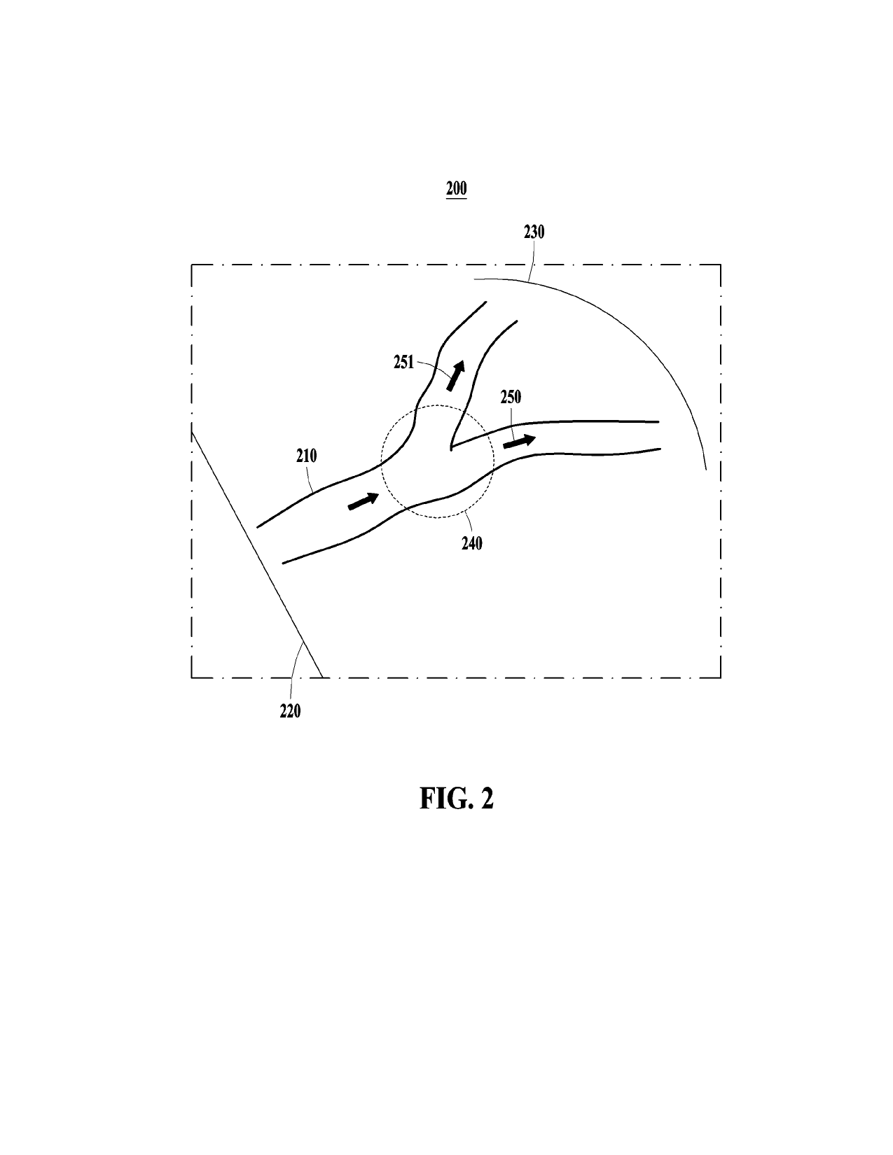

An electronic device 100 according to an embodiment is a device for processing a medical image (e.g., a medical image processing device) that analyzes blood vessels in the medical image. For example, the electronic device 100 may represent a device for quantitatively and qualitatively analyzing blood vessels included in a medical image. For example, the electronic device 100 may receive a two-dimensional (2D) or three-dimensional (3D) medical image acquired from a medical imaging device such as X-ray, computed tomography (CT), or magnetic resonance imaging (MRI) as an input, and analyze a blood vessel structure included in the image. For example, the electronic device 100 may identify blood vessels in the image. The electronic device 100 may analyze not only quantitative characteristics such as the diameter, length, and curvature of the blood vessels, but also qualitative characteristics such as branching structures of the blood vessels, presence or absence of stenosis, and morphological abnormalities. For example, the electronic device 100 may accurately determine whether a multi-branch point of a target blood vessel in the medical image is a bifurcation point that branches into two paths, a trifurcation point that branches into three paths, a quadrifurcation point that branches into four paths, or a pentafurcation point that branches into five paths. Branch points that may appear in the target blood vessel will be described in detail below with reference to FIGS. 2 to 5.

The electronic device 100 according to an embodiment may include an image acquisition unit 110, memory 120, and a processor 130.

For example, the electronic device 100 may acquire a medical image based on the image acquisition unit 110. The image acquisition unit 110 may acquire a medical image by directly capturing the medical image or may receive a medical image from an external device. For example, the image acquisition unit 110 may include hardware and/or software modules for acquiring a medical image including a blood vessel structure in a human body. For example, the image acquisition unit 110 may include a medical imaging device, such as a CT device, an MRI device, an angiography device, or an ultrasound device, but is not limited thereto. In another example, the image acquisition unit 110 may be connected to an external medical imaging device in a wired or wireless manner and receive a medical image from the external medical imaging device. For example, the image acquisition unit 110 may receive an X-ray image in Digital Imaging and Communications in Medicine (DICOM) format captured from an external C-arm device.

The memory 120 according to an embodiment may store a medical image acquired through the image acquisition unit 110. For example, the memory 120 may include a non-volatile storage device. For example, the memory 120 may include a hard disk drive (HDD), a solid state drive (SSD), or a flash memory. In another example, the memory 120 may include a volatile memory for temporary data processing. For example, the memory 120 may include dynamic random access memory (DRAM) or static random access memory (SRAM). However, this is only an example of the memory 120, and the memory 120 is not limited thereto. In addition, the memory 120 may store metadata (e.g., imaging date and time, patient information, imaging angle, etc.) corresponding to the medical image together. The memory 120 may store a plurality of instructions. For example, the memory 120 may include program code, algorithms, and control instructions executable by the processor 130.

The processor 130 according to an embodiment may execute a plurality of instructions included in the memory 120. For example, the processor 130 may analyze a blood vessel structure based on a medical image received from the image acquisition unit 110 by executing the plurality of instructions.

The processor 130 according to an embodiment may extract a target blood vessel from a medical image. For example, the processor 130 may extract a target blood vessel based on a user input. A user may select a start point and an end point of the blood vessel on the medical image. The processor 130 may extract a blood vessel path based on the start point and the end point of the blood vessel selected by the user. In another example, the processor 130 may automatically extract a target blood vessel. For example, the processor 130 may extract a target blood vessel by applying a filter (e.g., a Frangi filter) to the medical image. However, a method by which the processor 130 extracts a target blood vessel from a medical image is not limited thereto.

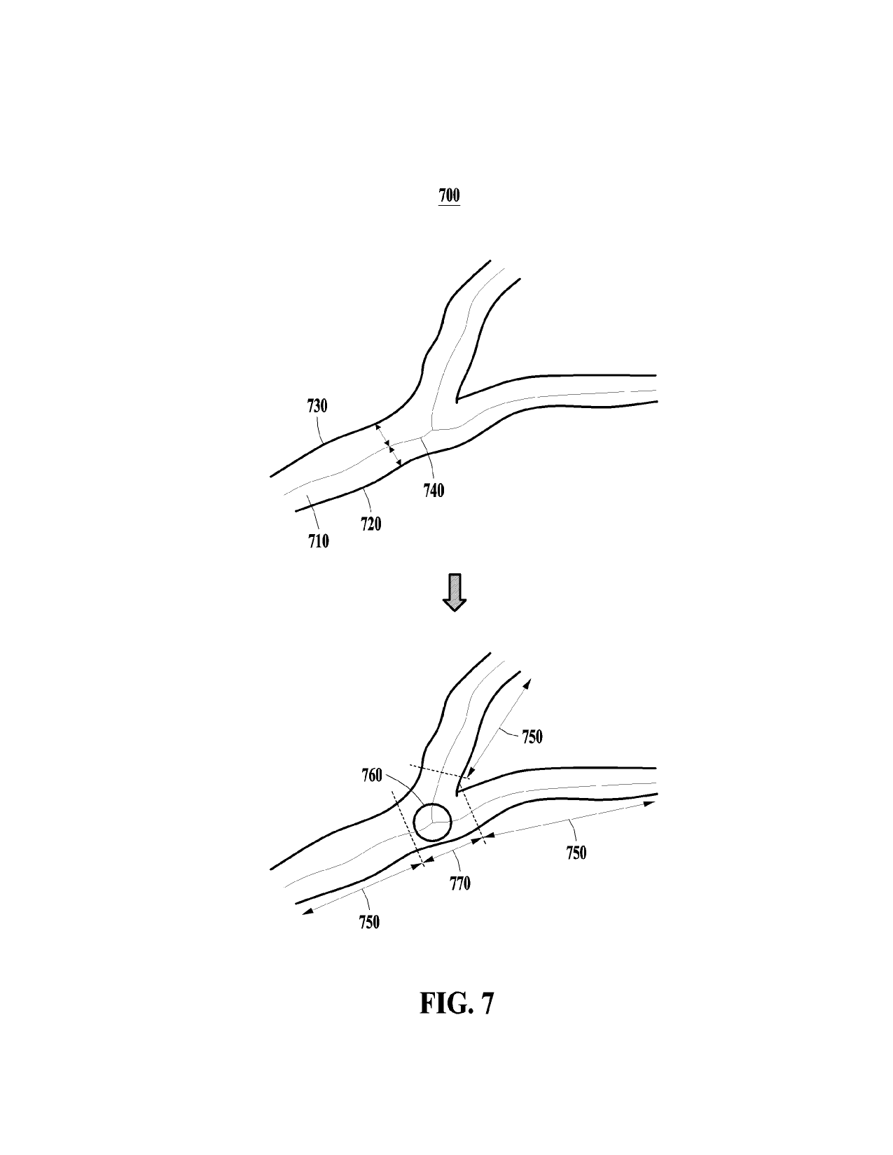

The processor 130 according to an embodiment may classify the extracted target blood vessel into a plurality of blood vessel sections. For example, the processor 130 may classify the target blood vessel into first blood vessel sections and second blood vessel sections including at least one branch point. For example, the first blood vessel sections represent sections that do not include a branch point. The second blood vessel sections represent sections that include one or more branch points. For example, the processor 130 may classify the plurality of blood vessel sections by scanning the target blood vessel. For example, the processor 130 may receive a predetermined first point and a predetermined second point on the target blood vessel. The first point and the second point may be selected by a user or may be automatically selected as a start point and an end point of a blood vessel to be analyzed. However, the predetermined first point and the predetermined second point are not limited thereto. The processor 130 may identify a branch point at which the target blood vessel branches to generate a plurality of blood vessel paths by scanning the target blood vessel from the first point toward the second point. The processor 130 may determine a section that generates a plurality of blood vessel paths among the target blood vessels as second blood vessel sections and determine the remaining sections as first blood vessel sections. In another example, the processor 130 may classify the target blood vessel into a plurality of blood vessel sections based on a vascular centerline corresponding to the target blood vessel. For example, the processor 130 may extract a vascular centerline corresponding to the target blood vessel. The processor 130 may extract the vascular centerline based on a contour of the target blood vessel. The processor 130 may generate a straight line (e.g., a vertical line) intersecting with a blood flow direction. The processor 130 may extract the vascular centerline based on a midpoint between outlines of the target blood vessel, forming intersection points with the straight line. The processor 130 may classify the second blood vessel sections including a point at which the vascular centerline branches. The processor 130 may classify the plurality of blood vessel sections as a tree structure. A method by which the processor 130 classifies the plurality of blood vessel sections as a tree structure is described in detail below with reference to FIG. 7.

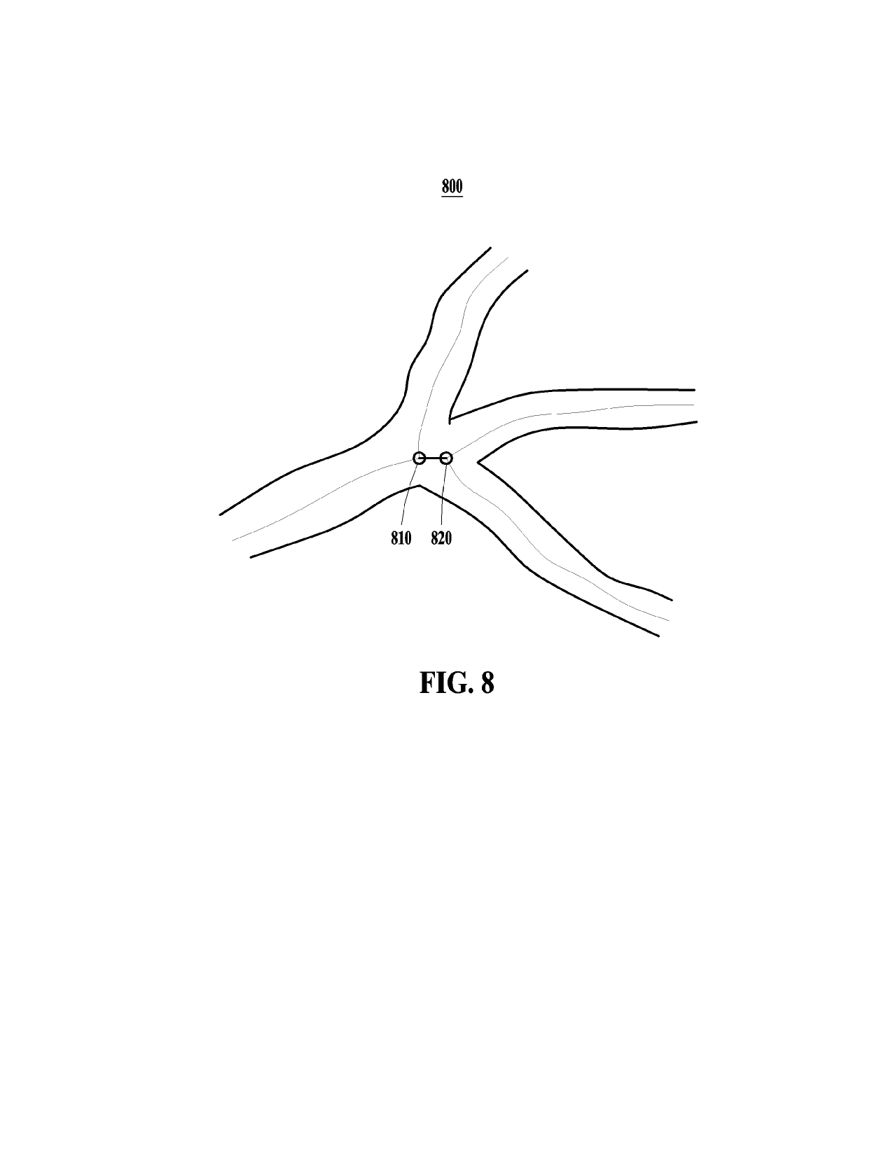

The processor 130 according to an embodiment may determine a potential multi-branch section among the plurality of second blood vessel sections. For example, the processor 130 may obtain a target section including a first branch point and a second branch point different from the first branch point among the second blood vessel sections. The processor 130 may calculate a first target length between the first branch point and the second branch point. The processor 130 may determine whether the target section is a potential multi-branch section based on the first target length. For example, the processor 130 may determine the target section as a potential multi-branch section when the first target length is less than a predetermined threshold length. A method by which the processor 130 determines a potential multi-branch section is described in detail below with reference to FIGS. 8 to 10.

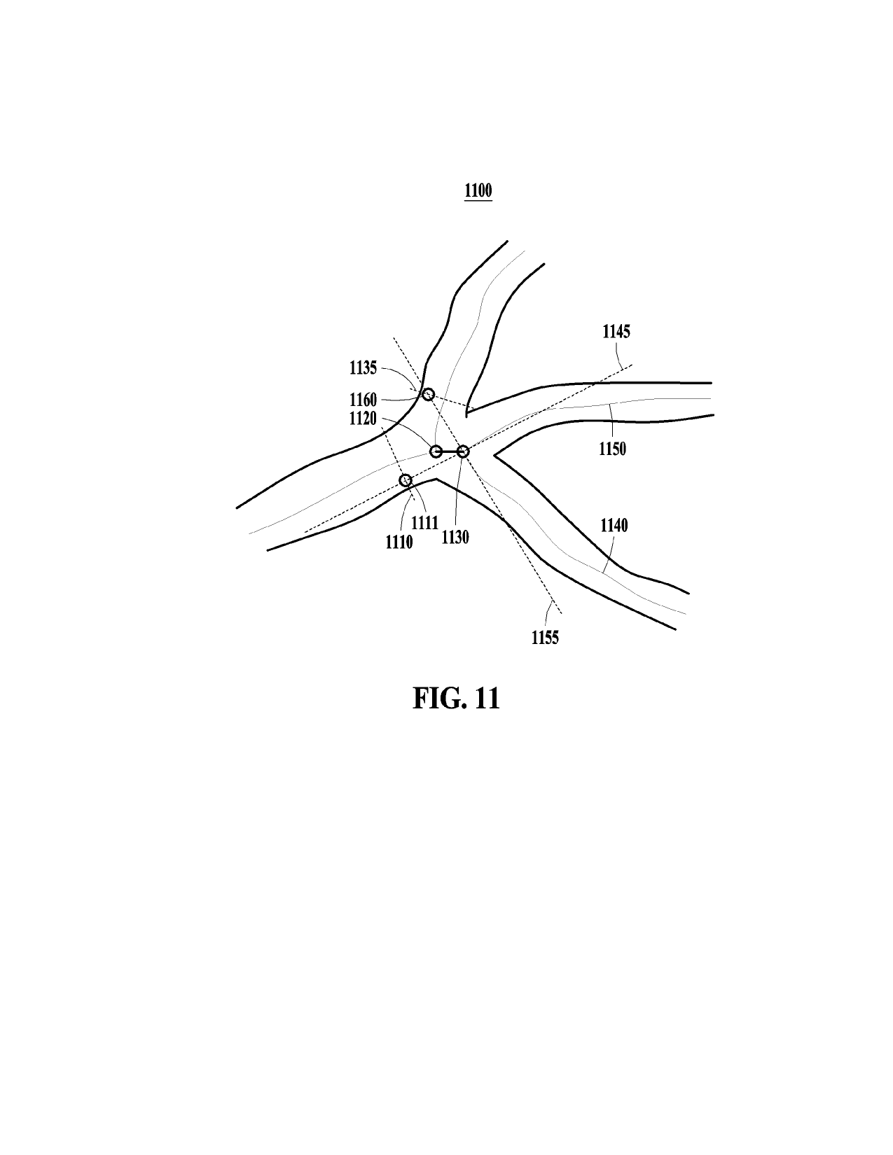

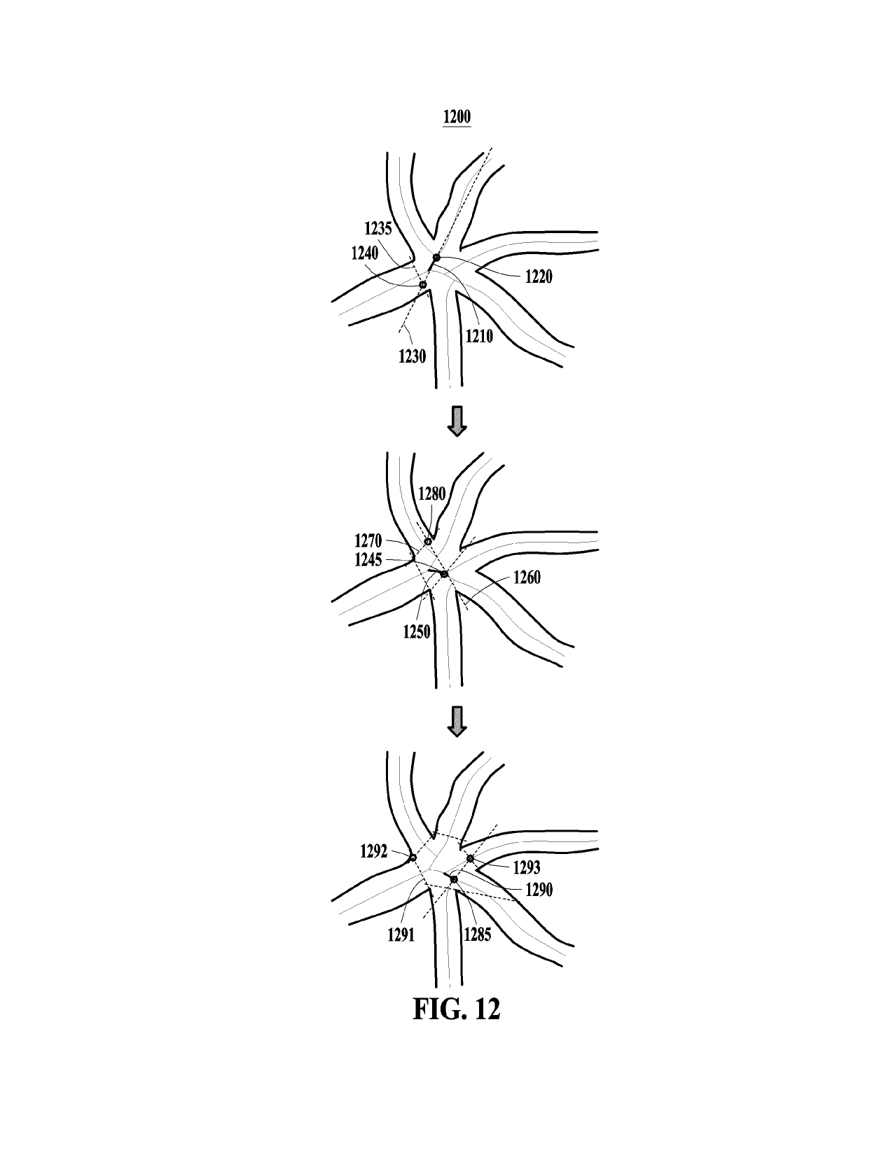

The processor 130 according to an embodiment may determine the potential multi-branch section as a target multi-branch section. For example, the processor 130 may generate a first straight line intersecting (e.g., perpendicularly intersecting) with the target blood vessel at a predetermined blood vessel point of the target section. For reference, the predetermined blood vessel point may correspond to a point located at a proximal portion among both end points of the target section. In another example, when the processor 130 classifies the plurality of blood vessel sections corresponding to the target blood vessel as a tree structure, the predetermined blood vessel point may represent a point at which the first blood vessel sections corresponding to higher-level nodes of the target section contact the target section. The predetermined blood vessel point will be described in detail below with reference to FIGS. 11 and 12. In addition, the processor 130 may generate a second straight line intersecting (e.g., perpendicularly intersecting) with the target blood vessel at the second branch point. The processor 130 may determine a position of an intersection point between the first straight line and the second straight line. The processor 130 may determine the potential multi-branch section as a target multi-branch section based on the position of the intersection point. For example, the processor 130 may determine the potential multi-branch section as the target multi-branch section when the intersection point is located within the target blood vessel.

The processor 130 according to an embodiment may provide vascular information corresponding to each of a plurality of paths connected to the target multi-branch section. For example, the processor 130 may provide vascular information, including a diameter, a curvature, and a branch angle of blood vessels on the plurality of blood vessel paths connected to the target multi-branch section. For example, the processor 130 may provide stent information, including at least one of information regarding the number of stents to be inserted into the plurality of paths and information regarding the sizes of the stents.

The electronic device 100 according to an embodiment may further include an actuator (not shown). In this case, the processor 130 may provide information regarding stent movement in the target multi-branch section by operating the actuator based on the vascular information corresponding to each of the plurality of paths connected to the target multi-branch section. The electronic device 100 may control which path of the target blood vessel the actuator moves the stent to based on the information regarding stent movement.

FIGS. 2 to 5 schematically illustrate branch types included in a target blood vessel according to an embodiment.

An electronic device according to an embodiment may extract a target blood vessel 210, 310, 410, or 510 from a medical image 200, 300, 400, or 500.

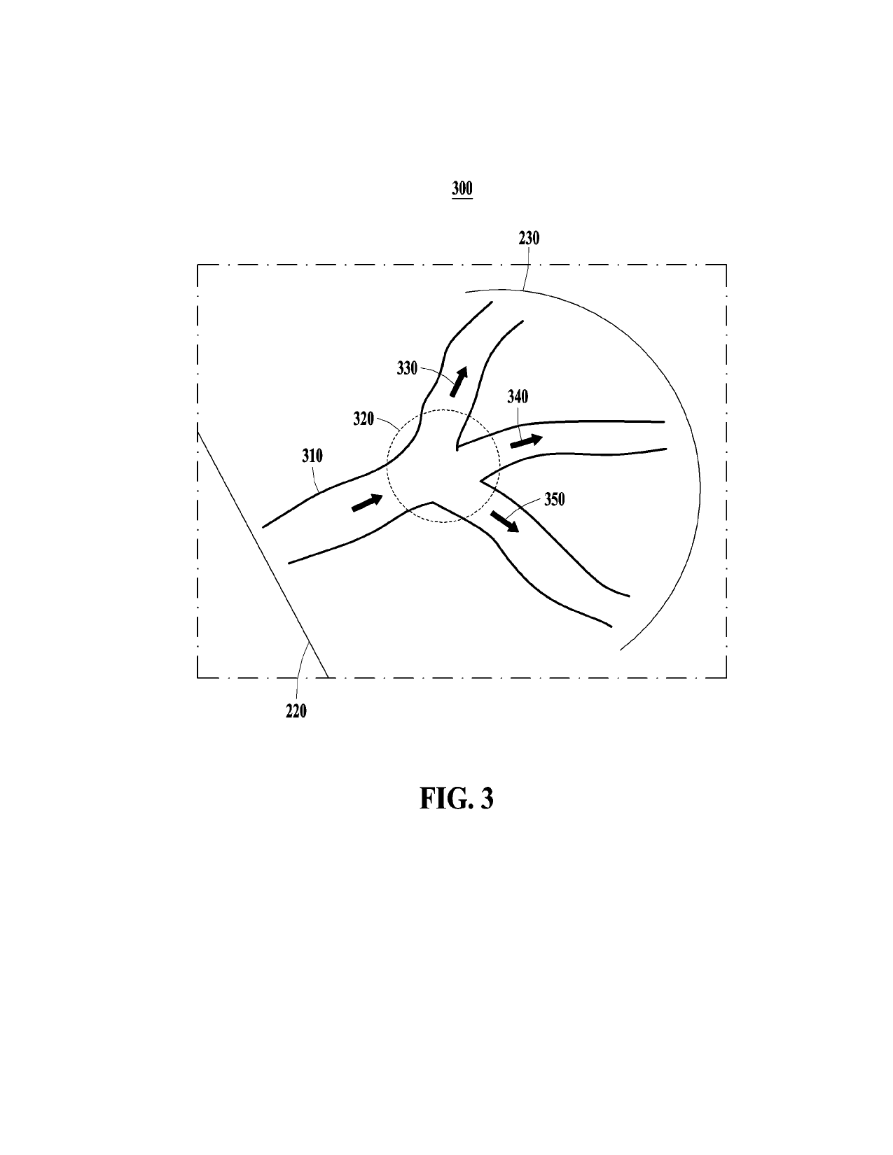

Referring to FIG. 2, a target blood vessel 210 extracted by the electronic device from a medical image 200 may include a branch point 240. For reference, the following description assumes that blood flow in the target blood vessel 210 flows from a proximal portion 220 toward a distal portion 230. The proximal portion 220 may represent a section corresponding to a starting point of the blood flow, and the distal portion 230 may represent a terminal section toward which the blood flow is directed. The proximal portion 220 and the distal portion 230 have the same meanings in FIGS. 3 to 5 below. The target blood vessel 210 may branch into a plurality of paths 250 and 251 at the branch point 240. The electronic device may detect the branch point 240 while scanning the target blood vessel 210 from the proximal portion 220 toward the distal portion 230. The electronic device may identify the branch point 240 at which the target blood vessel 210 branches to generate a plurality of blood vessel paths 250 and 251 by scanning the target blood vessel 210. FIG. 2 illustrates that the target blood vessel 210 branches into a first path 250 and a second path 251 at the branch point 240. A point branching into two different paths may be referred to as a bifurcation point. However, the name of the point is not limited thereto. The first path 250 and the second path 251 each represent a blood vessel extending from the branch point 240 toward the distal portion 230. For example, the first path 250 may represent a main blood vessel path, and the second path 251 may represent a branched auxiliary path, the first path 250 and the second path 251 are not limited thereto. The electronic device may scan each of the first and second paths 250 and 251 separated at the branch point 240 in parallel. The electronic device may detect other branch points included in the paths 250 and 251 by scanning the paths 250 and 251 in parallel. In other words, the electronic device may classify the blood vessel sections on all paths (e.g., the first path 250 and the second path 251) connected to the target blood vessel 210 into first blood vessel sections and second blood vessel sections.

Referring to FIG. 3, a target blood vessel 310 may branch into a plurality of paths 330, 340, and 350 at a branch point 320. For example, the target blood vessel 310 may branch into a first path 330, a second path 340, and a third path 350 at the branch point 320. As illustrated in FIG. 3, a point branching into three paths at the branch point 320 may be referred to as a trifurcation point. However, this is only an example, and the name of the point is not limited thereto. As described with reference to FIG. 2, the electronic device may detect the branch point 320 while scanning the target blood vessel 310 from the proximal portion 220 toward the distal portion 230. The electronic device may scan blood vessel paths 330, 340, and 350 extending toward the distal portion 230 from the branch point 320 in parallel. For example, when the electronic device identifies the branch point 320 while scanning the target blood vessel 310, the electronic device may simultaneously scan the first path 330, the second path 340, and the third path 350, branched at the branch point 320, and classify the blood vessel sections on each path.

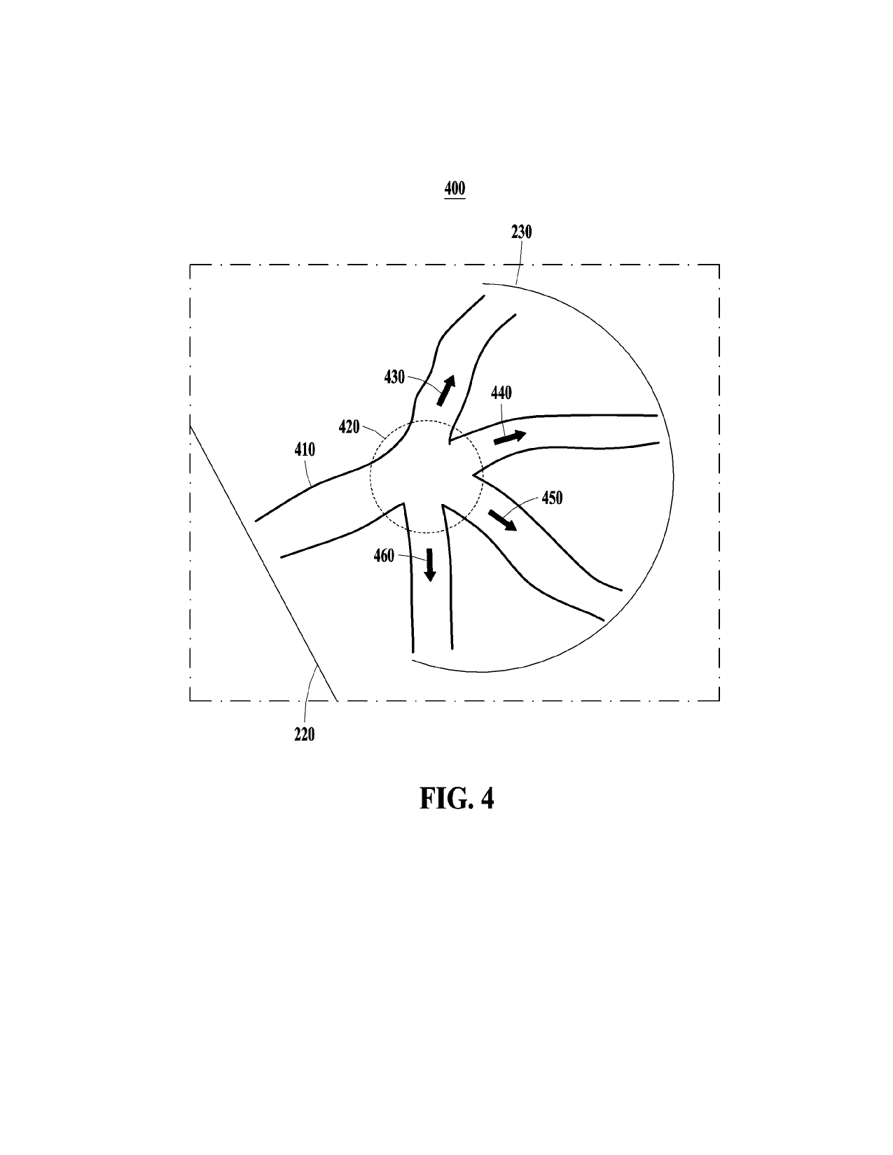

Referring to FIG. 4, a target blood vessel 410 extracted by the electronic device from a medical image 400 may include a branch point 420. The target blood vessel 410 may branch into a plurality of paths 430, 440, 450, and 460 at the branch point 420. The branch point 420 branching into four paths may be referred to as a quadrifurcation or tetrafurcation, but the name is not limited thereto. The electronic device may detect the branch point 420 while scanning the target blood vessel 410 from the proximal portion 220 toward the distal portion 230. The electronic device may classify the blood vessel sections in each path by scanning four paths generated at the branch point 420.

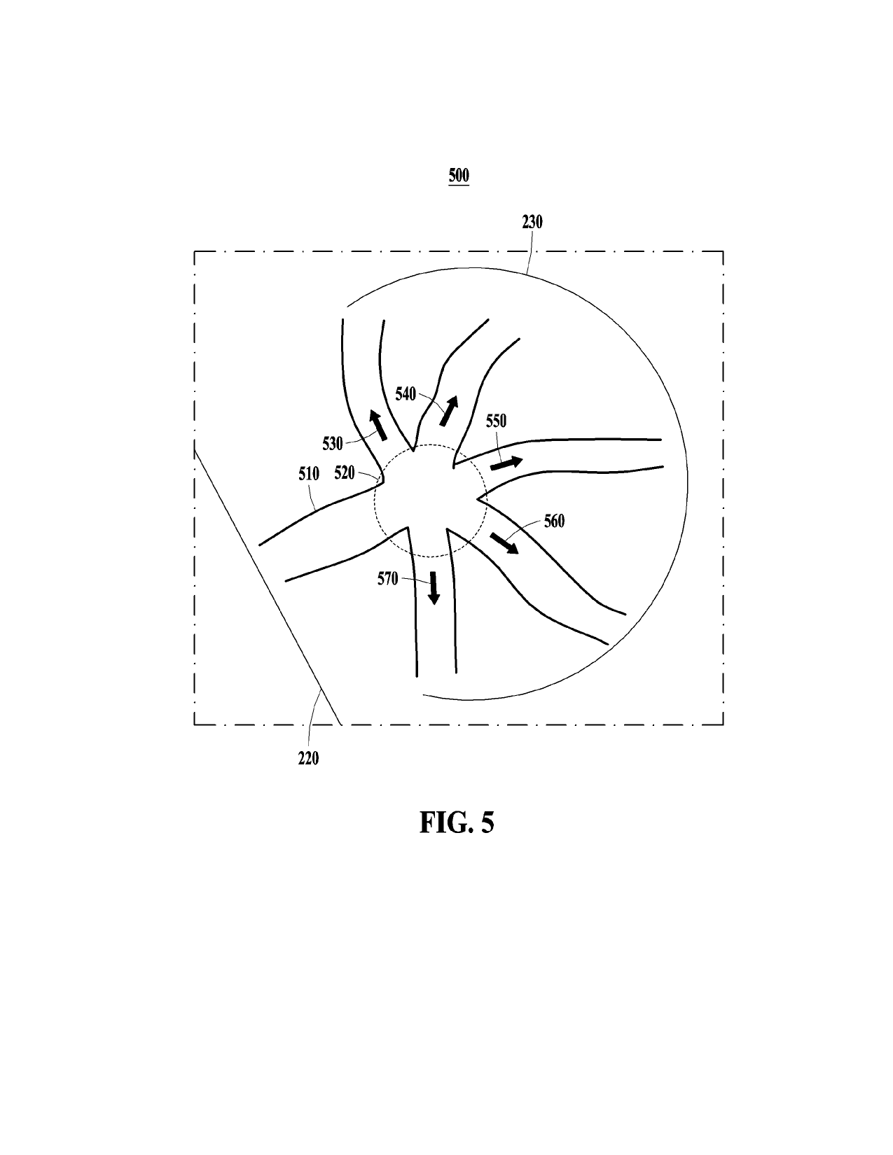

Referring to FIG. 5, a target blood vessel 510 extracted by the electronic device from a medical image 500 may include a branch point 520. The target blood vessel 510 may branch into a plurality of paths 530, 540, 550, 560, and 570 at the branch point 520. The branch point 520 branching into five paths may be referred to as a pentafurcation, but the name is not limited thereto. The electronic device may detect the branch point 520 while scanning the target blood vessel 510 from the proximal portion 220 toward the distal portion 230. The electronic device may subdivide the blood vessel sections in each path by scanning five paths generated at the branch point 520.

As described above with reference to FIGS. 2 to 5, the cases where the target blood vessel 210, 310, 410, or 510 includes a branch point 240, 320, 420, or 520 have been described. In FIGS. 2 to 5, the branch points are described as being distinguished by different names (e.g., a bifurcation, trifurcation, quadrifurcation, or pentafurcation point). However, the electronic device may designate a branch point as a ramification point regardless of the number of paths branching at the branch point. The electronic device may determine the branch point as a single ramification point and determine list information including elements corresponding to the number of paths connected to the branch point as information corresponding to the branch point. For example, referring to FIG. 2, when the electronic device identifies that two paths are connected at the branch point 240, the electronic device may determine the branch point 240 as a ramification point and store list information of size 2 to include elements corresponding to the first path 250 and the second path 251. In another example, referring to FIG. 3, when the electronic device identifies that three paths are connected at the branch point 320, the electronic device may determine the branch point 320 as a ramification point. The electronic device may store list information of size 3 to include elements corresponding to the first path 330, the second path 340, and the third path 350. In another example, referring to FIG. 4, when the electronic device identifies that four paths 430, 440, 450, and 460 are connected at the branch point 420, the electronic device may determine the branch point 420 as a ramification point. The electronic device may store list information of size 4 to include elements corresponding to each of the four paths 430, 440, 450, and 460. In another example, referring to FIG. 5, when the electronic device identifies that five paths 530, 540, 550, 560, and 570 are connected at the branch point 520, the electronic device may generate a list of size 5 and store list information to include elements corresponding to each of the five paths 530, 540, 550, 560, and 570. As such, the electronic device may dynamically generate a list according to the number of paths branching at a branch point. Each list may include information regarding paths connected to the corresponding branch point and may be used for blood vessel network analysis and visualization.

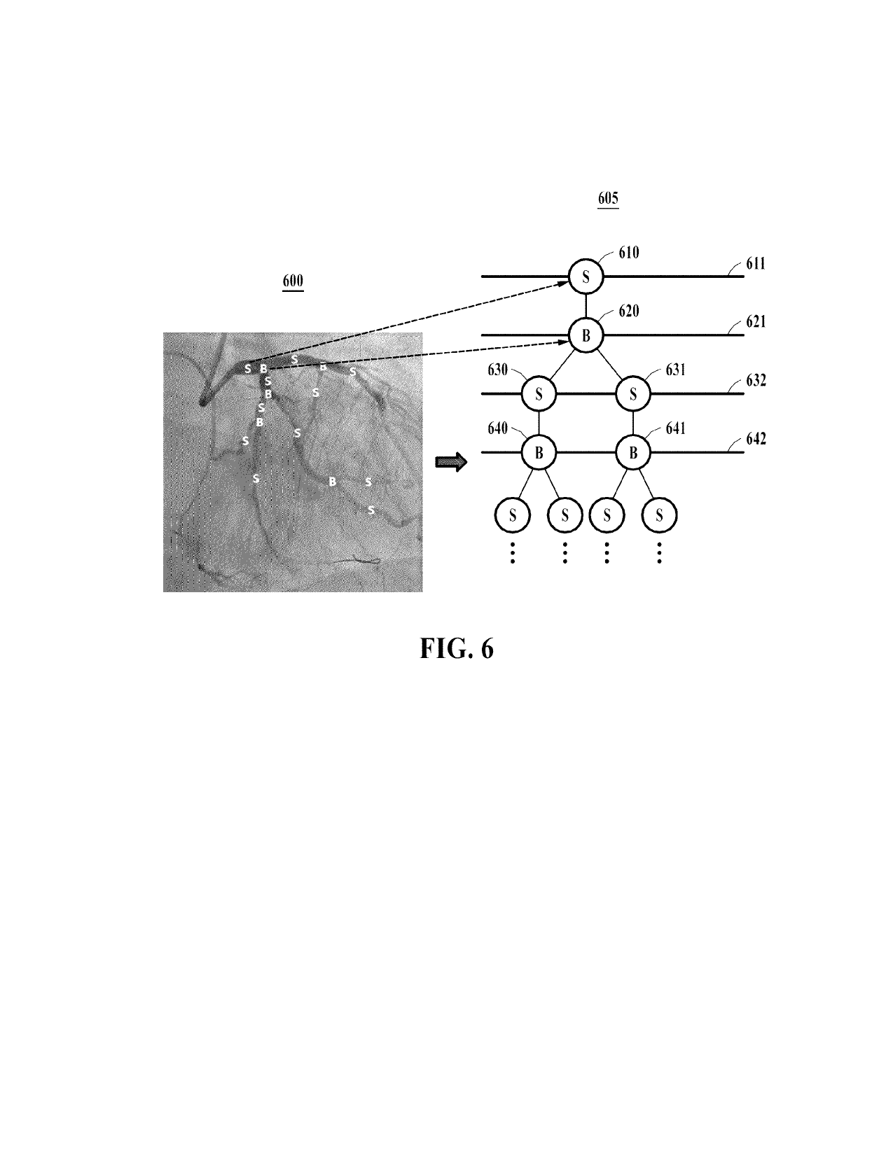

FIGS. 6 and 7 illustrate a method by which an electronic device classifies a target blood vessel into a plurality of blood vessel sections according to an embodiment.

Referring to FIG. 6, an electronic device may extract a target blood vessel from a medical image 600. The electronic device may analyze the extracted target blood vessel and classify the target blood vessel into a plurality of blood vessel sections. The electronic device may divide blood vessel sections corresponding to the target blood vessel into first blood vessel sections and second blood vessel sections. The first blood vessel section may represent a single path that does not include a branch point. The second blood vessel section may represent a section that includes one or more branch points. Referring to FIG. 6, the electronic device may display each blood vessel section in the target blood vessel in the medical image 600 by dividing the blood vessel sections into first blood vessel sections (e.g., S) and second blood vessel sections (e.g., B). For example, the electronic device may classify the first blood vessel sections and the second blood vessel sections as a tree structure 605.

Referring to FIG. 6, the tree structure 605 may hierarchically include the blood vessel sections (e.g., S and B). For reference, the tree structure 605 is a hierarchical data structure composed of nodes, and each node may be connected in a parent-child relationship. Here, a parent node and a child node are connected through an edge. For example, a node 610 may be connected as a parent node to a node 620, which is a child node. For example, a node 620 may be connected as a parent node to nodes 630 and 631, which are child nodes. In the tree structure 605, the node 610 is a root node representing the highest-level node of the tree structure 605. The tree structure 605 may be composed of multiple levels 611, 621, 632, and 642. The electronic device may set a root node (e.g., the node 610) as a first level 611, and the level may increase toward lower nodes. For example, in the tree structure 605, the first level 611 may include the node 610 corresponding to a starting point of the target blood vessel. The second level 621 may include the node 620 corresponding to a first branch point connected to the node 610. The third level 632 may include the nodes 630 and 631 connected to the node 620. The fourth level 642 may include child nodes 640 and 641 of the nodes 630 and 631 included in the third level 632. The electronic device may effectively identify a connection relationship and structure of the target blood vessel by classifying a plurality of blood vessel portions corresponding to the target blood vessel as the tree structure 605. FIG. 6 illustrates a case where the first blood vessel sections (e.g., S) and the second blood vessel sections (e.g., B) alternately appear in the target blood vessel. However, when the first blood vessel section connecting the second blood vessel section to another second blood vessel section is less than a predetermined threshold length, the electronic device may form the tree structure 605 such that nodes corresponding to different second blood vessel sections are consecutively connected.

Referring to FIG. 7, an electronic device may classify a target blood vessel into a plurality of blood vessel sections based on a vascular centerline 740. The electronic device according to an embodiment may extract a target blood vessel 710 from a medical image 700. For example, the electronic device may extract contours 720 and 730 corresponding to the target blood vessel 710. The electronic device may extract the vascular centerline 740 corresponding to the target blood vessel 710 based on the extracted contours 720 and 730. For example, the electronic device may extract intersection points between the contours 720 and 730 and a straight line perpendicular to the blood flow direction at a specific point of the target blood vessel 710. For example, the electronic device may extract intersection points between the contours 720 and 730 and a straight line perpendicular to the blood flow direction while scanning the target blood vessel 710 from a proximal portion toward a distal portion. The electronic device may calculate a midpoint of each of the extracted intersection points. The electronic device may extract the vascular centerline 740 corresponding to the target blood vessel 710 by connecting the midpoints of each of the intersection points.

The electronic device according to an embodiment may extract the vascular centerline 740 corresponding to the target blood vessel 710 and then scan the vascular centerline 740 from a predetermined first point to a predetermined second point. For example, the predetermined first point and the predetermined second point may be received from a user. For example, a user may determine the first point and the second point through an input (e.g., a click or location information input) for a point where analysis of the target blood vessel 710 in the medical image 700 is required. In another example, the electronic device may automatically determine the first point and the second point based on the blood flow direction in the target blood vessel 710. For example, the predetermined first point may correspond to a point closest to a proximal portion of the target blood vessel 710 in the medical image 700. In addition, the predetermined second point may correspond to a point closest to a distal portion of the target blood vessel 710 in the medical image 700. However, a method of predetermining the first point and the second point is not limited thereto. The electronic device may detect a branch point 760 at which the vascular centerline 740 branches by scanning the vascular centerline 740. For reference, the branch point 760 may represent a point at which the blood vessel branches into two or more paths. In other words, the branch point 760 may represent a section where the vascular centerline 740 divides into two or more while the electronic device scans the target blood vessel 710.

The electronic device according to an embodiment may classify the target blood vessel 710 into a plurality of blood vessel sections based on the detected branch point 760. For example, the electronic device may classify the target blood vessel 710 into a first blood vessel section 750 and a second blood vessel section 770. Here, the first blood vessel section 750 may represent a single path section that does not include the branch point 760. The second blood vessel section 770, which includes at least one branch point 760, may represent a region where the target blood vessel 710 begins to branch. The electronic device may classify a section including the branch point 760 as the second blood vessel section 770.

For reference, the first blood vessel section 750 and the second blood vessel section 770 illustrated in FIG. 7 may correspond to each node on the tree structure 605 described with reference to FIG. 6. For example, the electronic device may determine a node connected to a plurality of child nodes among the nodes on the tree structure 605 as a node corresponding to the second blood vessel section 770.

FIGS. 8 to 10 are diagrams for describing a method by which an electronic device determines a potential multi-branch section according to an embodiment.

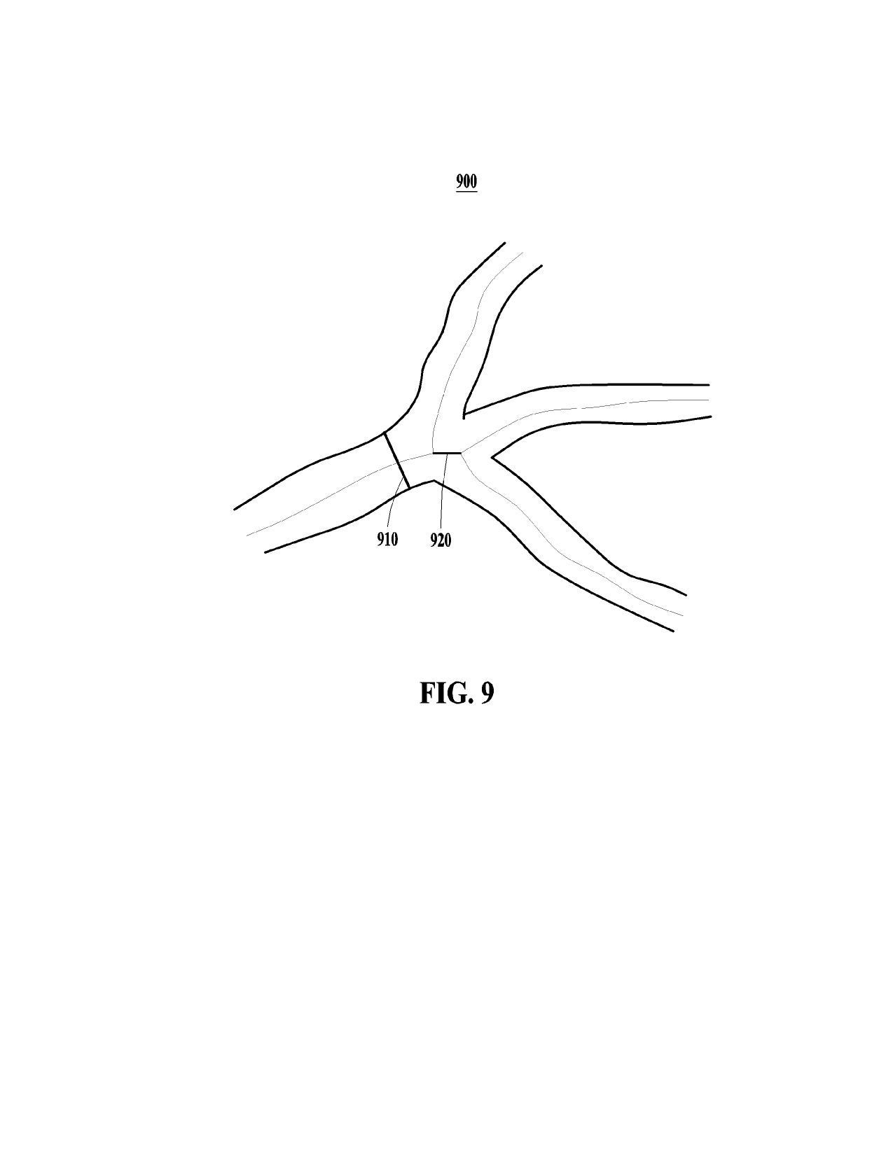

Referring to FIG. 8, an electronic device may determine a section including branch points 810 and 820 as a potential multi-branch section based on a distance between the branch points 810 and 820 included in a target blood vessel in a medical image 800. For example, the electronic device may extract a target blood vessel from the medical image 800. The electronic device may detect branch points 810 and 820 in the target blood vessel. For reference, when a target blood vessel branches into three or more paths, the target blood vessel may branch into three different paths at a single point on a vascular centerline or may branch into three different paths based on two consecutive branch points. Referring to FIG. 8, a case is illustrated where a target blood vessel branches into three different blood vessel paths based on a first branch point 810 and a second branch point 820, and the first branch point 810 and the second branch point 820 are located adjacent to each other. When the distance between the first branch point 810 and the second branch point 820 is very short (e.g., less than a predetermined threshold length), the electronic device may have difficulty classifying a section connecting the first branch point 810 to the second branch point 820 as one of a first blood vessel section and a second blood vessel section. In other words, when the distance between the first branch point 810 and the second branch point 820 is short, the electronic device may omit a section between the first branch point 810 and the second branch point 820 in a tree structure (e.g., the tree structure 605 of FIG. 6). Therefore, as described below with reference to FIG. 9, it is necessary to determine a potential multi-branch section including the first branch point 810 and the second branch point 820 by calculating the result of comparing a distance between the first branch point 810 and the second branch point 820 to a predetermined threshold length.

Referring to FIG. 9, an electronic device according to an embodiment may determine a potential multi-branch section based on a first target length 920 between a first branch point and a second branch point in a target blood vessel included in a medical image 900. For example, the electronic device may determine a point adjacent to a heart to which the target blood vessel is connected among both end points of a target section as a predetermined blood vessel point 910. For example, when the electronic device classifies the target blood vessel into first blood vessel sections and second blood vessel sections by scanning the target blood vessel from a proximal portion toward a distal portion, the predetermined blood vessel point 910 may represent a point at which a first blood vessel section and a second blood vessel section contact each other. FIG. 9 focuses on a case where the lower left of the target blood vessel is a proximal portion and the upper right is a distal portion. The electronic device may calculate the vascular diameter at the predetermined blood vessel point 910. For example, the electronic device may generate a straight line perpendicular to a vascular centerline at the predetermined blood vessel point 910. The electronic device may extract intersection points between the straight line and a contour of the target blood vessel. The electronic device may determine a distance between the extracted intersection points as the vascular diameter at the predetermined blood vessel point 910. The electronic device may calculate a threshold length based on the vascular diameter at the predetermined blood vessel point 910. For example, the electronic device may obtain the threshold length based on a result of an operation between the vascular diameter at the predetermined blood vessel point 910 and a predetermined threshold value. For example, the electronic device may determine a value obtained by dividing the vascular diameter at the predetermined blood vessel point 910 by the square root of 2 as the threshold length. In other words, the electronic device may determine one side of a square having the vascular diameter at the predetermined blood vessel point 910 as a diagonal as the threshold length. The electronic device may determine the target section as a potential multi-branch section based on the result of comparing the threshold length to the first target length 920. For example, the electronic device may classify the target section as a second blood vessel section when the first target length 920 is equal to or greater than the threshold length. In addition, the electronic device may determine the target section as a potential multi-branch section when the first target length 920 is less than the threshold length. In other words, the electronic device may determine the target section as a section branching into two blood vessel paths when the first target length 920 is equal to or greater than the threshold length. The electronic device may determine the target section as a section expected to branch into three or more blood vessel paths (e.g., a potential multi-branch section) when the first target length 920 is less than the threshold length.

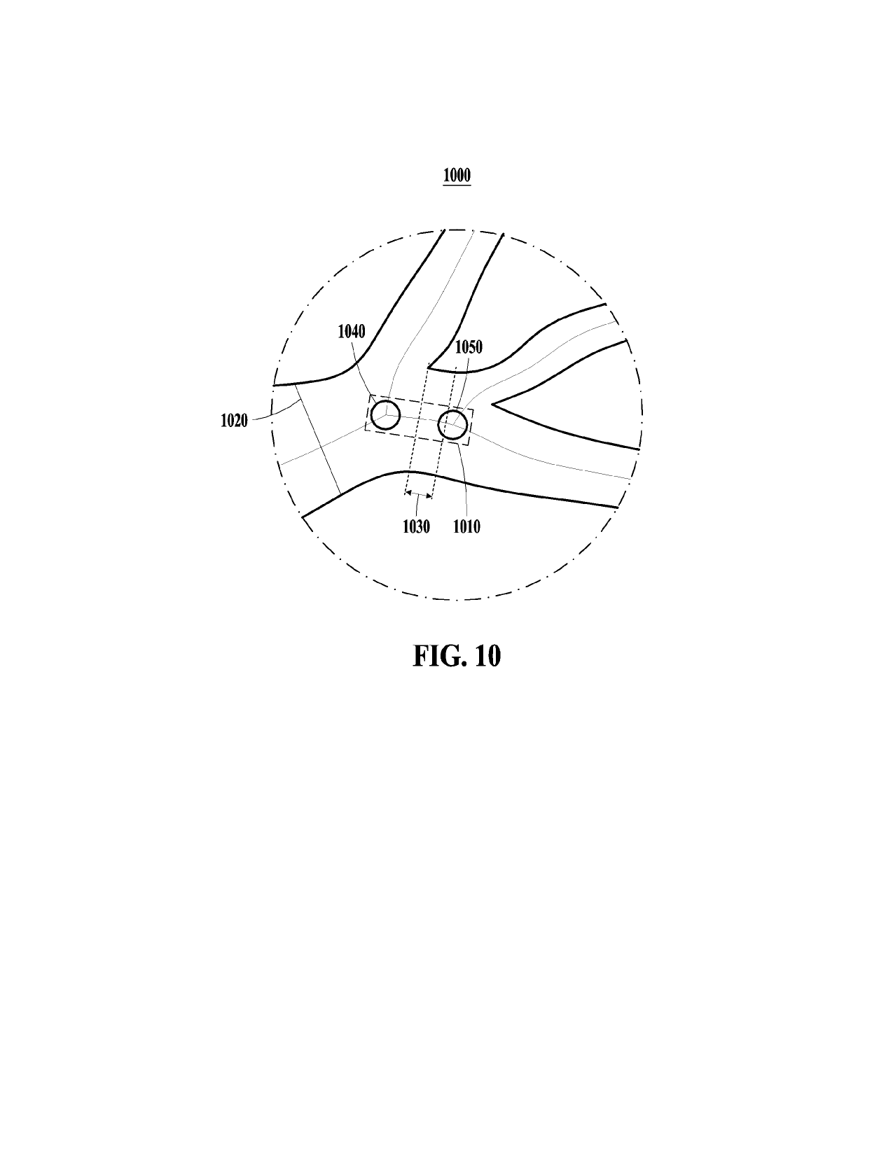

FIG. 10 illustrates a case where a first target length 1010 between a first branch point 1040 and a second branch point 1050 is equal to or greater than a threshold length 1030. FIG. 10 is a diagram for describing a method by which an electronic device classifies the blood vessel sections corresponding to a target blood vessel when the first target length 1010 is equal to or greater than the threshold length 1030. The electronic device may extract a target blood vessel from a medical image 1000. The electronic device may detect a first branch point 1040 and a second branch point 1050 in the extracted target blood vessel. The electronic device may calculate a first target length 1010 between the first branch point 1040 and the second branch point 1050. For reference, the first target length 1010 may represent the length of a centerline between two branch points, but is not limited thereto. The electronic device may compare the first target length 1010 to the threshold length 1030. Here, the threshold length 1030 may represent a value calculated based on the vascular diameter at a predetermined blood vessel point 1020. A method of obtaining the threshold length 1030 has been described with reference to FIG. 9, and thus a redundant description thereof is omitted.

When the first target length 1010 is equal to or greater than the threshold length 1030, the electronic device may define a section between the first branch point 1040 and the second branch point 1050 as a first blood vessel section. In addition, when the first target length 1010 is equal to or greater than the threshold length 1030, the electronic device may classify a section including the first branch point 1040 as a second blood vessel section and classify a section including the second branch point 1050 as a second blood vessel section. Therefore, when the first target length 1010 is equal to or greater than the threshold length 1030 calculated based on the predetermined blood vessel point 1020, the electronic device may determine that the target blood vessel illustrated in FIG. 10 has a structure in which a second blood vessel section including the first branch point 1040, a first blood vessel section between the first branch point 1040 and the second branch point 1050, and a second blood vessel section including the second branch point 1050 are connected. In other words, the electronic device may determine that the target blood vessel illustrated in FIG. 10 has a connection structure of "second blood vessel section - first blood vessel section - second blood vessel section" from the predetermined blood vessel point 1020. In this case, the electronic device may exclude the connection structure from the predetermined blood vessel point 1020 from a potential multi-branch section.

FIG. 11 is a diagram for schematically describing a method by which an electronic device determines a target multi-branch section according to an embodiment.

In FIG. 11, an electronic device may extract a target blood vessel from a medical image 1100. The electronic device may extract branch points (e.g., a first branch point 1120 and a second branch point 1130) from the extracted target blood vessel. The electronic device may determine a section including a plurality of branch points in the target blood vessel as a target section. End points of the target section may represent points in contact with a first blood vessel section (e.g., a blood vessel section that does not include a branch point). For example, the end points of the target section may represent points at which a first blood vessel section and a second blood vessel section contact each other. The electronic device may generate first straight lines 1110 and 1135 intersecting with the target blood vessel at predetermined blood vessel points of the target section. Here, the predetermined blood vessel points represent end points of the target section. For example, assuming a case where the electronic device classifies each section of the target blood vessel as a plurality of tree structures (e.g., the tree structure 605 of FIG. 6), the electronic device may obtain nodes of a higher level than a node corresponding to the target section in the tree structure. The electronic device may determine a point in contact with the target section among both end points of each of the first blood vessel sections corresponding to the obtained higher-level nodes as a predetermined blood vessel point. The electronic device may generate first straight lines 1110 and 1135 at each of the predetermined blood vessel points. In addition, the electronic device may generate second straight lines 1145 and 1155 intersecting with the target blood vessel at the second branch point 1130. Here, the second straight lines 1145 and 1155 may represent straight lines perpendicular to vascular centerlines 1140 and 1150 respectively corresponding to a plurality of paths. However, the second straight lines 1145 and 1155 are not limited thereto and may represent straight lines perpendicular to the vascular centerlines 1140 and 1150 generated at predetermined intervals from the second branch point 1130 in a direction of the vascular centerlines 1140 and 1150. In other words, the electronic device may generate second straight lines 1145 and 1155 perpendicular to the vascular centerlines 1140 and 1150 at predetermined intervals from the second branch point 1130 along the vascular centerlines 1140 and 1150. The electronic device may determine positions of intersection points 1111 and 1160 between the first straight lines 1110 and 1135 and the second straight lines 1145 and 1155. The electronic device may determine the potential multi-branch section as a target multi-branch section based on the positions of the intersection points 1111 and 1160. The electronic device may determine whether a position of at least one of the intersection points 1111 and 1160 is within the target blood vessel. For example, when an intersection point 1111 between the first straight line 1110 and the second straight line 1145 is located outside the target blood vessel, the electronic device may determine the potential multi-branch section as a second blood vessel section. In other words, when the intersection point 1111 is located outside the target blood vessel, the electronic device may determine that the potential multi-branch section is a section branching into two paths (e.g., a bifurcation section) and does not correspond to a multi-branch section. When the intersection point 1111 is located within the target blood vessel, the electronic device may determine the potential multi-branch section as a target multi-branch section. When the electronic device determines the potential multi-branch section as a target multi-branch section, the electronic device may combine a section including the first branch point 1120 and a section including the second branch point 1130 into a single node on a tree structure (e.g., the tree structure 605 of FIG. 6). In other words, the electronic device may combine a plurality of branch points (e.g., the first branch point 1120 and the second branch point 1130) located within the target multi-branch section and determine the combined branch points as a single multi-branch point (e.g., a ramification point).

FIG. 12 illustrates a specific method by which an electronic device determines a target multi-branch section in a target blood vessel according to an embodiment.

Referring to FIG. 12, an electronic device may extract a target blood vessel from a medical image 1200. Assuming that the target blood vessel illustrated in FIG. 12 includes four different paths, like the target blood vessel illustrated in FIG. 4, a method by which the electronic device determines a target multi-branch section in the target blood vessel is described below.

The electronic device according to an embodiment may classify a target blood vessel into first blood vessel sections and second blood vessel sections. The electronic device may extract a target section located between an arbitrary first blood vessel section and another first blood vessel section. When the target section includes a first branch point, a second branch point 1220 located at a distal portion of the target blood vessel relative to the first branch point, and a third branch point 1245 located at a distal portion of the target blood vessel relative to the second branch point 1220, the electronic device may sequentially compare distances between the branch points to a threshold length. For reference, a method of calculating the threshold length has been described with reference to FIG. 9, and thus a redundant description thereof is omitted. For example, the electronic device may compare a first target length 1210 to the threshold length. The electronic device may determine a section including the first target length 1210 as a potential multi-branch section only when the first target length 1210 between the first branch point and the second branch point 1220 is less than the threshold length. Thereafter, the electronic device may compare a second target length 1250 between the third branch point 1245 and an adjacent branch point (e.g., the first branch point or the second branch point 1220) to the threshold length. The electronic device may determine a section including the second target length 1250 as a potential multi-branch section only when the second target length 1250 is less than the threshold length. The electronic device may compare a third target length 1290 corresponding to a fourth branch point 1285 to the threshold length. The electronic device may determine a section including the third target length 1290 as a potential multi-branch section only when the third target length 1290 is less than the threshold length. In other words, the electronic device may determine whether lengths between branch points (e.g., the second to fourth branch points 1220, 1245, and 1285) are less than the threshold length, determine a section as an independent branch section (e.g., a second blood vessel section) when the length is equal to or greater than the threshold length, and determine a section as a potential multi-branch section when the length is less than the threshold length.

The electronic device according to an embodiment may determine a potential multi-branch section and then determine whether each section included in the potential multi-branch section is a target multi-branch section. First, the electronic device may generate a first straight line 1235 intersecting with the target blood vessel at a predetermined blood vessel point. The electronic device may also generate a second straight line 1230 intersecting with the target blood vessel at the second branch point 1220. Here, the second straight line 1230 represents a straight line perpendicular to a vascular centerline at the second branch point 1220. The electronic device may determine a position of an intersection point 1240 between the first straight line 1235 and the second straight line 1230. When the determined intersection point 1240 is located within the target blood vessel, the electronic device may determine the potential multi-branch section including the first target length 1210 as a target multi-branch section. Thereafter, the electronic device may generate a third straight line 1260 intersecting with the target blood vessel at the third branch point 1245. The electronic device may generate straight lines (e.g., the first straight line 1235 and a straight line 1270) perpendicular to the target blood vessel at end points of the potential multi-branch section. The electronic device may determine a position of an intersection point 1280 between the third straight line 1260 and straight lines (e.g., the first straight line 1235 and the straight line 1270) perpendicular to the target blood vessel at end points of the potential multi-branch section. The electronic device may determine whether the position of the intersection point 1280 is located within the target blood vessel, and when the intersection point 1280 is located within the target blood vessel, the electronic device may determine the potential multi-branch section including the second target length 1250 as a target multi-branch section. Subsequently, the electronic device may generate a fourth straight line intersecting with the target blood vessel at the fourth branch point 1285. The fourth straight line may represent a straight line perpendicular to a vascular centerline corresponding to the target blood vessel at the fourth branch point 1285. The electronic device may determine whether an intersection point 1293 between the fourth straight line and straight lines at end points of the potential multi-branch section (e.g., including a straight line 1291 as straight lines perpendicular to a vascular centerline corresponding to the target blood vessel at boundary points of the potential multi-branch section) is located within the target blood vessel. When the intersection point 1293 is located within the target blood vessel, the electronic device may determine the potential multi-branch section including the third target length 1290 as a target multi-branch section.

In summary, the electronic device may detect a section (or region) of the target blood vessel including a plurality of branch points. The electronic device may determine whether distances between the branch points included in the section are less than a threshold distance and determine a potential multi-branch section to include branch points determined to be less than the threshold distance. The electronic device may identify an intersection point between a straight line perpendicular to the target blood vessel at a boundary point of the potential multi-branch section and a straight line perpendicular to a vascular centerline corresponding to the target blood vessel at each of the branch points included in the potential multi-branch section. When the position of the identified intersection point is located within the target blood vessel, the electronic device may determine the potential multi-branch section as a target multi-branch section. Therefore, the electronic device may sequentially analyze a section including multiple branch points and determine whether the section corresponds to a target multi-branch section. Here, the target multi-branch section may be referred to as a ramification section. The ramification section may correspond to a data structure in list format. For example, when the target blood vessel branches into two paths, the electronic device may define a target multi-branch section of list size 2. In another example, when the target blood vessel branches into N paths, the electronic device may define a target multi-branch section of list size N. Here, N represents a natural number of 2 or greater.

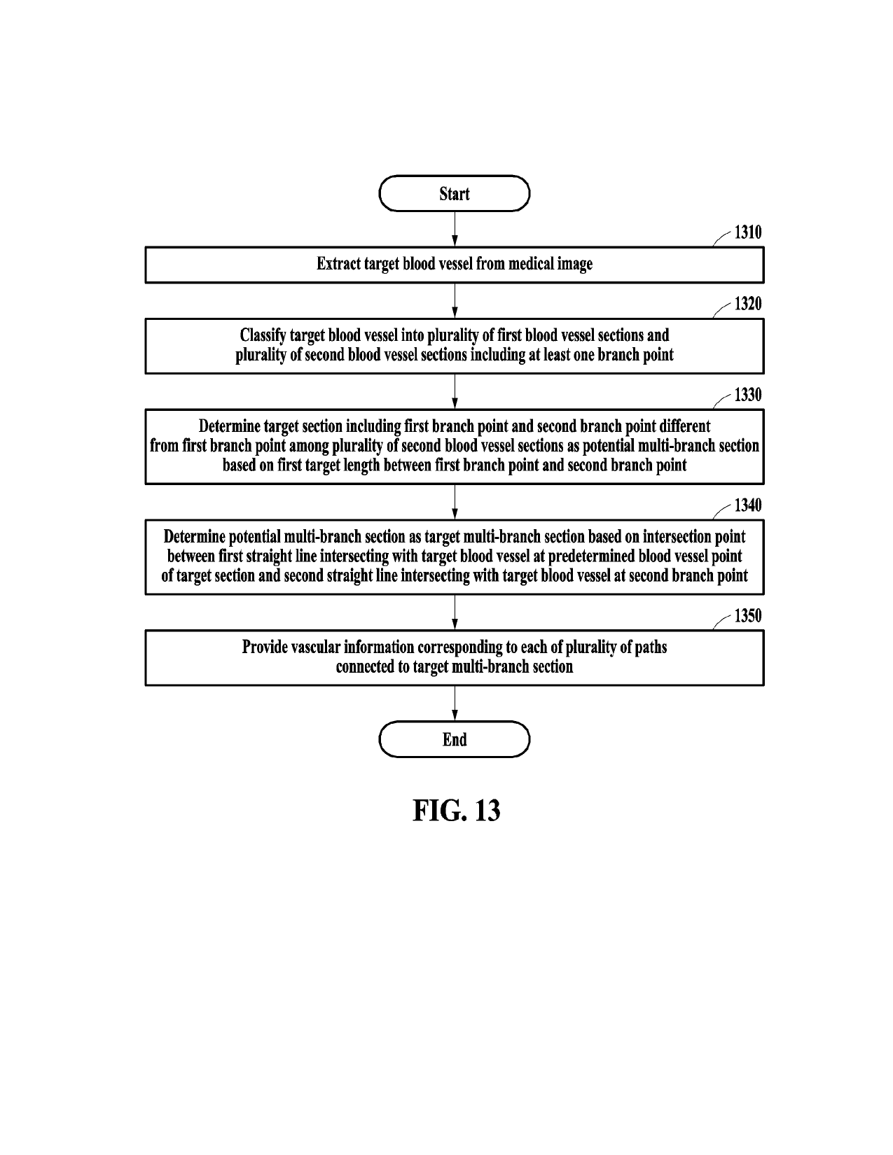

FIG. 13 is a flowchart schematically illustrating a method performed by an electronic device according to an embodiment.

Referring to FIG. 13, a process in which an electronic device according to an embodiment analyzes a target blood vessel in a medical image and determines a multi-branch section is described step by step. In each step, the electronic device may perform tasks of extracting a target blood vessel from a medical image, classifying blood vessel sections, and detecting a multi-branch section.

In operation 1310, the electronic device according to an embodiment may extract a target blood vessel from a medical image. The electronic device may receive medical image data such as X-ray, CT, or MRI as an input. The electronic device may remove background noise from the received medical image through filtering and preprocessing. The electronic device may extract an outline of a blood vessel using a contour extraction algorithm. The electronic device may generate a vascular centerline based on the extracted contour. The electronic device may use the generated vascular centerline as a reference line for subsequent analysis.

In operation 1320, the electronic device according to an embodiment may classify the target blood vessel into a plurality of first blood vessel sections and a plurality of second blood vessel sections including at least one branch point. The electronic device may detect a branch point in the blood vessel by performing scanning along the vascular centerline. The electronic device may divide the blood vessel into multiple sections based on the branch point. The electronic device may classify a section that does not include a branch point as a first blood vessel section. The electronic device may classify a section including at least one branch point as a second blood vessel section.

In operation 1330, the electronic device according to an embodiment may identify a section including a first branch point and a second branch point different from the first branch point among the plurality of second blood vessel sections. The electronic device may calculate the centerline length between the first branch point and the second branch point (e.g., the first target lengths 920 and 1010 in FIGS. 9 and 10). The electronic device may compare the calculated first target length to a predetermined threshold length. The electronic device may determine the section as a potential multi-branch section when the first target length is less than the threshold length.

In operation 1340, the electronic device according to an embodiment may generate a first straight line intersecting with the target blood vessel at a predetermined blood vessel point of a target section. The electronic device may generate a second straight line intersecting with the target blood vessel at the second branch point. The electronic device may calculate an intersection point between the first straight line and the second straight line. The electronic device may determine whether the intersection point is within the target blood vessel. The electronic device may determine the potential multi-branch section as a target multi-branch section when the intersection point is located within the blood vessel.

In operation 1350, the electronic device according to an embodiment may provide vascular information corresponding to each of a plurality of paths connected to the target multi-branch section. The electronic device may calculate the diameter of each of the plurality of paths connected to the target multi-branch section. The electronic device may calculate the length and the curvature of each path. The electronic device may calculate information regarding the sizes and numbers of stents that can be inserted into each path. The electronic device may provide vascular information to medical staff, and the medical staff may use the vascular information for diagnosis and planning for the target blood vessel.

The embodiments described herein may be implemented using a hardware component, a software component, and/or a combination thereof. For example, the devices, the methods, and the components described in the embodiments may be implemented using a general-purpose or special-purpose computer, such as a processor, a controller and an arithmetic logic unit (ALU), a digital signal processor (DSP), a microcomputer, a field programmable gate array (FPGA), a programmable logic unit (PLU), a microprocessor, or any other devices capable of responding to and executing instructions. The processing device may run an operating system (OS) and software applications that run on the OS. The processing device also may access, store, manipulate, process, and create data in response to execution of the software. For purpose of simplicity, the description of a processing device is singular; however, one of ordinary skill in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or one or more combinations thereof, to independently or collectively instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

As used herein, "A or B", "at least one of A and B", "at least one of A or B", "A, B or C", "at least one of A, B and C", and "at least one of A, B, or C," each of which may include any one of the items listed together in the corresponding one of the phrases, or all possible combinations thereof.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, structure, device, or circuit are combined in a different manner, and/or replaced or supplemented by other components or their equivalents.

Therefore, other implementations, other embodiments, and equivalents to the claims are also within the scope of the following claims.

What is claimed is:

1. An electronic device for medical image processing, the electronic device comprising:
an image acquisition unit configured to acquire a medical image;
memory configured to store the medical image and a plurality of instructions; and
a processor configured to execute the plurality of instructions included in the memory,
wherein the processor is configured to extract a target blood vessel from the medical image, classify the target blood vessel into a plurality of first blood vessel sections and a plurality of second blood vessel sections including at least one branch point, determine a target section including a first branch point and a second branch point different from the first branch point among the plurality of second blood vessel sections as a potential multi-branch section based on a first target length between the first branch point and the second branch point, determine the potential multi-branch section as a target multi-branch section based on an intersection point between a first straight line intersecting with the target blood vessel at a predetermined blood vessel point of the target section and a second straight line intersecting with the target blood vessel at the second branch point, and provide vascular information corresponding to each of a plurality of paths connected to the target multi-branch section.

2. The electronic device of claim 1, wherein the processor is configured to determine a point adjacent to a heart to which the target blood vessel is connected among both end points of the target section as the predetermined blood vessel point, and determine the target section as the potential multi-branch section based on a result of comparing the first target length to a threshold length calculated based on a vascular diameter at the predetermined blood vessel point.

3. The electronic device of claim 2, wherein the processor is configured to obtain the threshold length based on a result of an operation between the vascular diameter at the predetermined blood vessel point and a predetermined threshold value, classify the target section as the second blood vessel section when the first target length is equal to or greater than the threshold length, and determine the target section as the potential multi-branch section when the first target length is less than the threshold length.

4. The electronic device of claim 1, wherein the processor is configured to classify the plurality of first blood vessel sections and the plurality of second blood vessel sections as a tree structure, obtain nodes of a higher level than a node corresponding to the target section in the tree structure, generate the first straight line at each of predetermined blood vessel points corresponding to points in contact with the target section among both end points of each of first blood vessel sections corresponding to the obtained nodes, and determine the potential multi-branch section as the target multi-branch section based on the intersection point between the first straight line and the second straight line.

5. The electronic device of claim 1, wherein the processor is configured to determine whether the intersection point between the first straight line and the second straight line is located within the target blood vessel, determine the potential multi-branch section as the second blood vessel section when the intersection point is located outside the target blood vessel, and determine the potential multi-branch section as the target multi-branch section when the intersection point is located within the target blood vessel.

6. The electronic device of claim 1, wherein the processor is configured to,
when the target section includes the first branch point, the second branch point located at a distal portion of the target blood vessel relative to the first branch point, and a third branch point located at a distal portion of the target blood vessel relative to the second branch point,
determine the target section as the potential multi-branch section based on a second target length between the second branch point and the third branch point, and determine the potential multi-branch section as the target multi-branch section based on an intersection point between the first straight line and a third straight line intersecting with the target blood vessel at the third branch point.

7. The electronic device of claim 1, wherein the processor is configured to classify the plurality of first blood vessel sections and the plurality of second blood vessel sections as a tree structure.

8. The electronic device of claim 1, wherein the processor is configured to provide stent information including at least one of information regarding a number of stents to be inserted into the plurality of paths and information regarding sizes of the stents, based on the vascular information corresponding to each of the plurality of paths connected to the target multi-branch section.

9. The electronic device of claim 1, further comprising an actuator, wherein the processor is configured to control stent movement in the target multi-branch section by operating the actuator based on the vascular information corresponding to each of the plurality of paths connected to the target multi-branch section.

10. The electronic device of claim 1, wherein the processor is configured to receive a predetermined first point and a predetermined second point on the target blood vessel, identify a branch point at which the target blood vessel branches to generate a plurality of blood vessel paths by scanning the target blood vessel from the first point toward the second point, and classify the target blood vessel into the plurality of first blood vessel sections and the plurality of second blood vessel sections based on parallel scanning of the plurality of blood vessel paths.

11. The electronic device of claim 1, wherein the processor is configured to extract a vascular centerline corresponding to the target blood vessel, detect points at which the vascular centerline branches as branch points while scanning the vascular centerline from a predetermined first point to a predetermined second point, and classify a section including at least one branch point among the branch points as the second blood vessel section.

12. A method of processing a medical image, performed by an electronic device, the method comprising:
extracting a target blood vessel from a medical image;
classifying the target blood vessel into a plurality of first blood vessel sections and a plurality of second blood vessel sections including at least one branch point;
determining a target section including a first branch point and a second branch point different from the first branch point among the plurality of second blood vessel sections as a potential multi-branch section based on a first target length between the first branch point and the second branch point;
determining the potential multi-branch section as a target multi-branch section based on an intersection point between a first straight line intersecting with the target blood vessel at a predetermined blood vessel point of the target section and a second straight line intersecting with the target blood vessel at the second branch point; and
providing vascular information corresponding to each of a plurality of paths connected to the target multi-branch section.

13. The method of claim 12, wherein the determining of the target section as the potential multi-branch section comprises:
determining a point adjacent to a heart to which the target blood vessel is connected among both end points of the target section as the predetermined blood vessel point; and
determining the target section as the potential multi-branch section based on a result of comparing the first target length to a threshold length calculated based on a vascular diameter at the predetermined blood vessel point.

14. The method of claim 13, wherein the determining of the target section as the potential multi-branch section comprises:
obtaining the threshold length based on a result of an operation between the vascular diameter at the predetermined blood vessel point and a predetermined threshold value;
classifying the target section as the second blood vessel section when the first target length is equal to or greater than the threshold length; and
determining the target section as the potential multi-branch section when the first target length is less than the threshold length.

15. The method of claim 12, wherein the determining of the potential multi-branch section as the target multi-branch section comprises:
classifying the plurality of first blood vessel sections and the plurality of second blood vessel sections as a tree structure;
obtaining nodes of a higher level than a node corresponding to the target section in the tree structure;
generating the first straight line at each of predetermined blood vessel points corresponding to points in contact with the target section among both end points of each of first blood vessel sections corresponding to the obtained nodes; and
determining the potential multi-branch section as the target multi-branch section based on the intersection point between the first straight line and the second straight line.

16. The method of claim 12, wherein the determining of the potential multi-branch section as the target multi-branch section comprises:
determining whether the intersection point between the first straight line and the second straight line is located within the target blood vessel;
determining the potential multi-branch section as the second blood vessel section when the intersection point is located outside the target blood vessel; and
determining the potential multi-branch section as the target multi-branch section when the intersection point is located within the target blood vessel.

17. The method of claim 12, wherein the determining of the potential multi-branch section as the target multi-branch section comprises:
when the target section includes the first branch point, the second branch point located at a distal portion of the target blood vessel relative to the first branch point, and a third branch point located at a distal portion of the target blood vessel relative to the second branch point,
determining the target section as the potential multi-branch section based on a second target length between the second branch point and the third branch point; and
determining the potential multi-branch section as the target multi-branch section based on an intersection point between the first straight line and a third straight line intersecting with the target blood vessel at the third branch point.

18. The method of claim 12, wherein the classifying of the target blood vessel into the plurality of first blood vessel sections and the plurality of second blood vessel sections comprises classifying the plurality of first blood vessel sections and the plurality of second blood vessel sections as a tree structure.

19. The method of claim 12, wherein the providing of the vascular information comprises providing stent information including at least one of information regarding a number of stents to be inserted into the plurality of paths and information regarding sizes of the stents, based on the vascular information corresponding to each of the plurality of paths connected to the target multi-branch section.

20. A non-transitory computer-readable storage medium storing one or more computer programs including instructions for performing the method of claim 12.
